(19) United States
(12) Patent Application Publication
(10) Pub. No.: US 2022/0139744 A1
(43) Pub. Date: May 5, 2022

(54) WAFER DEFECT ANALYSIS METHOD AND SYSTEM, DEVICE AND MEDIUM

(71) Applicant: CHANGXIN MEMORY TECHNOLOGIES, INC., Hefei (CN)

(72) Inventor: MENG-HSUAN TSAI, Hefei (CN)

(21) Appl. No.: 17/569,570

(22) Filed: Jan. 6, 2022

Related U.S. Application Data
(63) Continuation of application No. PCT/CN2021/098753, filed on Jun. 7, 2021.

(30) Foreign Application Priority Data
Jun. 8, 2020 (CN) 202010514472.4

(51) Int. Cl. H01L 21/67; G01N 25/72

(52) U.S. Cl. CPC H01L 21/67288; G01N 2033/0095; G01N 25/72

(57) ABSTRACT

Provided are a wafer defect analysis method and system, a device and a medium. The wafer defect analysis method includes: acquiring batch information and defect information of each wafer in a semiconductor manufacturing process, the defect information including hot spot defect information; setting a hot spot defect feature, and selecting target hot spot defect information associated with the hot spot defect feature from the hot spot defect information; tracking, according to the batch information, a first wafer corresponding to the target hot spot defect information associated with the hot spot defect feature, and determining a defect source.

### CROSS-REFERENCE TO RELATED APPLICATION(S)

This application is continuation of international application PCT/CN2021/098753, filed on Jun. 7, 2021, which refers to Chinese patent application No. 202010514472.4, filed on Jun. 8, 2020 and entitled “WAFER DEFECT ANALYSIS METHOD AND SYSTEM, DEVICE AND MEDIUM”. The contents of international application PCT/CN2021/098753 and Chinese patent application No. 202010514472.4 are hereby incorporated by reference in their entireties.

### TECHNICAL FIELD

The disclosure relates to the technical field of semiconductor manufacturing, and in particular to a wafer defect analysis method and system, a device and a medium.

### BACKGROUND

With the rapid development of the semiconductor integrated circuit, its manufacturing process has become more and more complex. At present, the advanced integrated circuit manufacturing process typically needs to complete hundreds of process steps in a plurality of process devices, and gradually forms a plurality of process films on a wafer through these process steps, and finally forms a required circuit structure. Since the wafer is transferred among different process devices, it is vulnerable to contamination of foreign particles, resulting in circuit pattern defects, which seriously affects the yield of products. The technique for detecting foreign particles or pattern defects on the wafers using detection systems such as an optical measurement detection system has been widely used, and these detection systems for monitoring the foreign particles or defects provide data support for fault analysis. However, under the limitation of the time period of current fault analysis, it can only be determined that there is a defect spot on a certain batch of wafers, and which wafers have been affected, but the source of the defect spot cannot be tracked.

### SUMMARY

In a first aspect, the embodiments of the disclosure provide a wafer defect analysis method, which includes: batch information and defect information of each wafer in a semiconductor manufacturing process are acquired, the defect information including hot spot defect information; a hot spot defect feature is set, and target hot spot defect information associated with the hot spot defect feature is selected from the hot spot defect information; a first wafer corresponding to the target hot spot defect information associated with the hot spot defect feature is tracked according to the batch information, and a defect source is determined.

In a second aspect, the embodiments of the disclosure further provide a wafer defect analysis system, which includes: an information acquisition module configured to acquire batch information and defect information of each wafer in a semiconductor manufacturing process, the defect information including hot spot defect information; a target defect information filtering module configured to select, according to a set hot spot defect feature, target hot spot defect information associated with the hot spot defect feature from the hot spot defect information; and a wafer tracking module configured to track, according to the batch information, a first wafer corresponding to the target hot spot defect information associated with the hot spot defect feature, and determine a defect source.

In a third aspect, the embodiments of the disclosure further provide an electronic device. The electronic device includes: one or more processor; and a storage device configured to store one or more programs. When the one or more programs are executed by the one or more processors, the one or more processors implements any wafer defect analysis method of an embodiment of the present disclosure.

In a fourth aspect, the embodiments of the disclosure further provide a computer readable storage medium, on which a computer program is stored. When the program is executed by a processor, any wafer defect analysis method of an embodiment of the disclosure is implemented.

### BRIEF DESCRIPTION OF THE DRAWINGS

FIG. 1 is a flowchart of a wafer defect analysis method provided by an embodiment of the disclosure.

FIG. 2 is a schematic diagram of a wafer defect analysis method provided by an embodiment of the disclosure.

FIG. 3 is a schematic diagram of another wafer defect analysis method provided by an embodiment of the disclosure.

FIG. 4 is a schematic diagram of yet another wafer defect analysis method provided by an embodiment of the disclosure.

FIG. 5 is a flowchart of a wafer defect analysis method provided by another embodiment of the disclosure.

FIG. 6 is a schematic diagram of a wafer defect analysis method provided by another embodiment of the disclosure.

FIG. 7 is a schematic diagram of another wafer defect analysis method provided by another embodiment of the disclosure.

FIG. 8 is a schematic structural diagram of a wafer defect analysis system provided by yet another embodiment of the disclosure.

FIG. 9 is a schematic structural diagram of another wafer defect analysis system provided by yet another embodiment of the disclosure.

FIG. 10 is a schematic structural diagram of an electronic device provided by still another embodiment of the disclosure.

### DETAILED DESCRIPTION

The following detailed description will be further described below with reference to the drawings and embodiments. It will be appreciated that the specific embodiments described herein are only used to explain the disclosure, instead of limiting the disclosure. Further, it will also be noted that in order to facilitate the description, only parts of the structure associated with the disclosure rather than all structures are shown in the drawings.

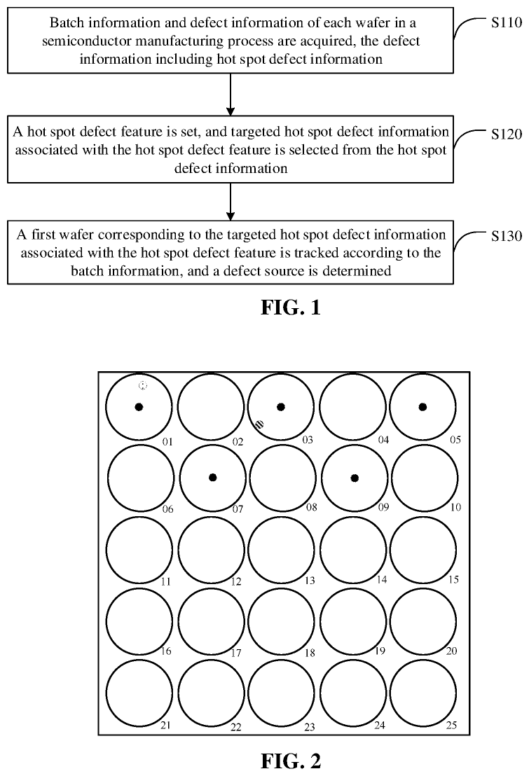

FIG. 1 is a flowchart of a wafer defect analysis method provided by an embodiment of the disclosure. As shown in FIG. 1, the wafer defect analysis method includes the following operations.

At S110, batch information and defect information of each wafer in a semiconductor manufacturing process are acquired, the defect information including hot spot defect information.

A wafer is a basic raw material for manufacturing semiconductor devices. The semiconductor of extremely high purity is prepared into the wafer by steps of crystal pulling and slicing, and then a tiny circuit structure is formed through a series of semiconductor manufacturing processes, which then becomes a chip after cutting, encapsulating, testing. The chip is widely applied to various electronic devices. During the process of semiconductor manufacturing, after a plurality of process steps is performed on the wafer surface, a desired circuit structure is finally formed on the wafer surface. During the process, each wafer needs to go through a plurality of process sites, and a corresponding process film is manufactured in each of the process sites.

The surface of a wafer chuck, which carries the wafer in a process device machine, could be contaminated caused by work environment or external factors, thereby causing contamination to the wafer surface. When a defect test is performed on the contaminated wafer, it is found that a hot spot defect appears on the wafer surface. Such a hot spot defect that is caused by a wafer backside particle is a key abnormal situation in the manufacturing process. During a conventional hot spot defect detection, when it is detected that three or five wafers in the same batch that are consecutively operated on the same wafer chuck have hot spot abnormalities occurred at the same location, it is determined that the hot spot abnormalities are contaminated by the wafer chuck surface. The hot spot defect is a wafer chuck defect. The tracking of wafers with such defect is limited to the production process of the same batch of wafers. Before a lithography machine automatically removes contaminants on the surface of a supporting stage when the machine confirms that the hot spot defect is the wafer chuck defect, there are already more than or equal to three or five wafers in a batch having the hot spot abnormalities occurred at the surface since they are contaminated by the surface of the supporting stage.

In a mass production process, since a plurality of batches of wafers uses the same wafer chuck in the same process device, the defects that are contaminated by the wafer chuck may exist in different batches of wafers. If only the wafer defects in the same batch are monitored while the continuity of chuck contamination is overlooked, it not only will cause misjudgment of the wafer defects, but also will not be beneficial to subsequent fault analysis, resulting in difficulty in finding the source of the fault. In the embodiment, the manufacturing information of each wafer is collected to generate a database. The database includes at least the batch information including a number of each wafer, a batch number of each wafer, a machine number and a wafer chuck number, and the hot spot defect information obtained from detection after corresponding process steps are executed on each wafer surface.

At S120, a hot spot defect feature is set, and target hot spot defect information associated with the hot spot defect feature is selected from the hot spot defect information.

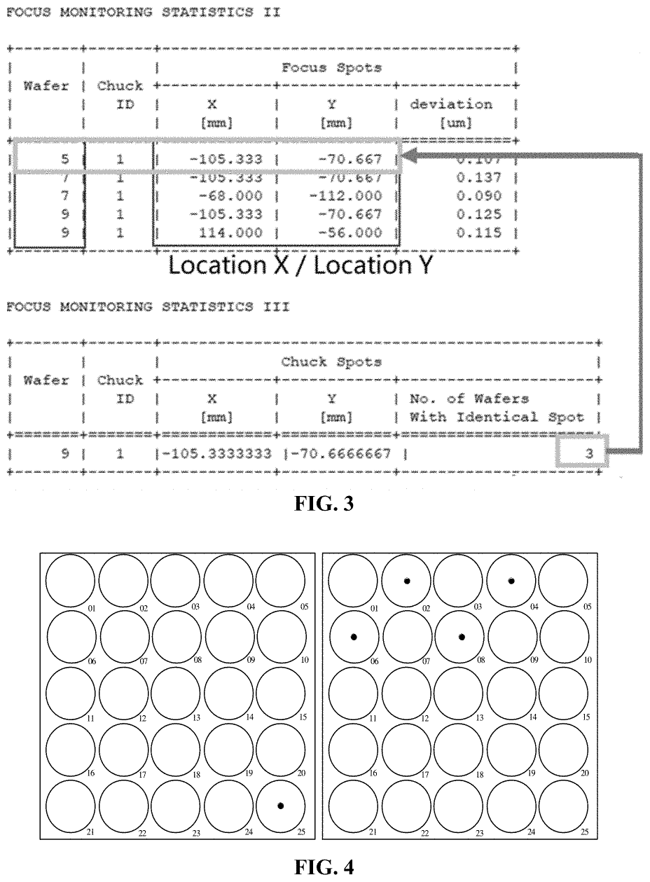

After the batch information and the defect information of each wafer in the manufacturing process are acquired, the target hot spot defect information associated with the hot spot defect feature is found from the acquired hot spot defect information according to the set hot spot defect feature. In conjunction with FIG. 2 and FIG. 3, for example, the quantity of the chucks carrying the wafers in a machine is two. In the 25 wafers in the same batch, the ID numbers of the wafers are 01, 02, . . . , 25, respectively, and the 25 wafers enter the two chucks respectively, for example, the wafers, indicated by ID numbers 01, 03, . . . , 23, 25, enter the chuck indicated by a chuck ID 001, and the wafers, indicated by ID numbers 02, 04, . . . , 22, 24, enter the chuck indicated by a chuck ID 002. When at least three or more wafers are consecutively located on the same chuck ID, and the hot spot defect information occurs at the same location information, it can be determined that the hot spot defect information is the target hot spot defect information. As shown in FIG. 3, after the wafers, indicated by ID numbers 01, 03, . . . , 23, 25, respectively enter the chuck indicated by the chuck ID 001, a defect at location coordinates (−105.333, −70.667) occurs on each of the wafers indicated by ID numbers 05, 07, and 09. At this time, it is determined that the defect information whose location information is (−105.333, −70.667) is the target hot spot defect information.

It is to be noted that FIG. 2 and FIG. 3 schematically illustrate that the set hot spot defect feature is location coordinates, or may be set as other hot spot defect feature. The embodiments of the disclosure do not limit the set hot spot defect feature.

Further, in the target hot spot defect information determined according to the hot spot defect feature, the wafers that consecutively occur with respect to the same chuck ID and the same location information may be in the same batch or different batches, thereby achieving acquisition of wafer defect data among different batches.

Further, the target hot spot defect information includes horizontal and vertical coordinates of a wafer surface hot spot.

It is to be noted that a contaminated point of the wafer chuck surface directly affects the back surface of the wafer and indirectly affect the wafer surface. When the wafer defect information is detected, the defect information of the front and back surfaces of the wafer may be detected.

At S130, a first wafer corresponding to the target hot spot defect information associated with the hot spot defect feature is tracked according to the batch information, and a defect source is determined.

When the hot spot defect information on different wafers appears at the same chuck ID and the same location at least three times, it may be determined that the hot spot defect information is the determined target hot spot defect information. Then, according to the batch information of each wafer, the first wafer corresponding to the target hot spot defect information that is associated with the hot spot defect feature is tracked and the wafer is determined as the defect source. For example, as shown in FIG. 4, the quantity of the chucks carrying wafers in the machine is two. The first chuck that carries the wafers has an ID of 001, and the second chuck that carries the wafers has an ID of 002. There are 25 wafers in the first batch. The wafers, indicated by ID numbers 01, 03, . . . , 23, 25, enter the chuck indicated by chuck ID 001, and the wafers, indicated by ID numbers 02, 04, . . . , 22, 24, enter the chuck indicated by chuck ID 002. There are 25 wafers in the second batch. The wafers indicated by ID numbers 01, 03, . . . , 23, 25, enter into chuck indicated by chuck ID 002, and the wafers, indicated by ID numbers 02, 04, . . . , 22, 24, enter the chuck indicated by chuck ID 001. Since the hot spot defect information occurs at a surface of the 25th wafer among the first batch of manufactured wafers, on the wafers indicated by ID numbers 02, 04, 06, and 08 in the second batches, hot spot defects occur at a location the same as that at the 25th wafer of the first batch. The 25th wafer in the first batch and the wafers indicated by ID numbers 02, 04, 06, and 08 of the second batch all go through the chuck indicated by ID 001. Since there always exist three or more wafers of the adjacent batches on the same chuck ID, and hot spot defect information occurs at the same location information, at this time it may be determined that the hot spot defect information is the target hot spot defect information. Therefore, it may be determined that the wafer is the defect source by tracking the first wafer corresponding to the target hot spot defect information associated with the hot spot defect feature and the batch where the first wafer is located.

According to the embodiments of the disclosure, the batch information and the hot spot defect information of the wafer in the manufacturing process are acquired, the target hot spot defect information is selected according to the hot spot defect feature, and finally the first wafer corresponding to the target hot spot defect information is tracked according to the batch information and the defect source is determined, so as to improve the accuracy for tracking the wafer defect source by.

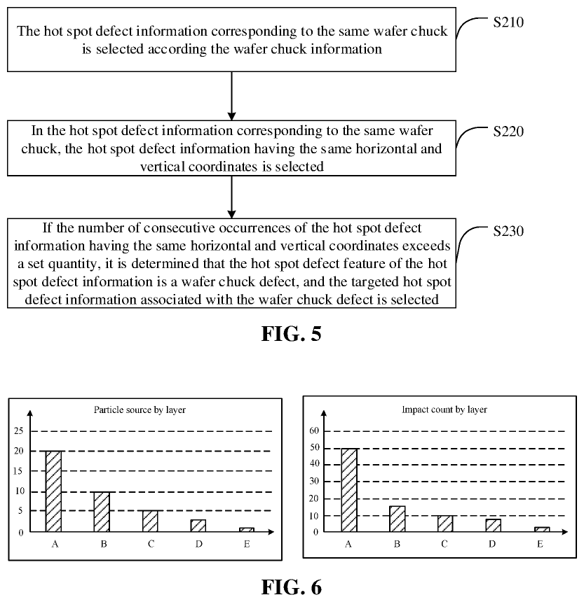

In an embodiment, based on the above-mentioned embodiments, FIG. 5 is a flowchart of a wafer defect analysis method provided by another embodiment of the disclosure. As shown in FIG. 5, the batch information of each wafer includes wafer chuck information corresponding to each wafer. The operation of selecting the target hot spot defect information associated with the hot spot defect feature from the hot spot defect information includes the following operations.

At S210, the hot spot defect information corresponding to the same wafer chuck is selected according the wafer chuck information.

For example, the quantity of the chucks carrying the wafers in the machine is two. In the same batch, the 25 wafers are indicated by ID numbers 01, 02, . . . , 25, respectively. The 25 wafers enter the two chucks respectively. For example, the wafers, indicated by ID numbers 01, 03, . . . , 23, 25, enter the chuck indicated by chuck ID 001, and the wafers, indicated by ID numbers 02, 04, . . . , 22, 24, enter the chuck indicated by chuck ID 002. In the 25 wafers of the same batch, the wafers indicated by ID numbers 01, 03, . . . , 23, 25 go through the chuck indicated by the same chuck ID, and the wafers indicated by ID numbers 02, 04, . . . , 22, 24 go through the chuck indicated by the same chuck ID. That is, the hot spot defect information corresponding to the same wafer chuck may be selected according to the acquired wafer chuck ID information.

At S220, in the hot spot defect information corresponding to the same wafer chuck, the hot spot defect information having the same horizontal and vertical coordinates is selected.

After the hot spot defect information corresponding to the same wafer chuck ID is selected, the hot spot defect information having the same horizontal and vertical coordinates is selected according to the horizontal and vertical coordinates of the acquired wafer surface hot spot. For example, when the wafer chuck IDs are the same, defects with horizontal and vertical coordinates of (−105.333, −70.667) occur to the chuck corresponding to wafers indicated by ID numbers 05, 07, and 09. At this time, it is determined the hot spot information whose horizontal and vertical coordinates are (−105.333, −70.667) is the hot spot defect information having the same horizontal and vertical coordinates.

At S230, if the number of consecutive occurrences of the hot spot defect information having the same horizontal and vertical coordinates exceeds a set quantity, it is determined that the hot spot defect feature of the hot spot defect information is a wafer chuck defect, and the target hot spot defect information associated with the wafer chuck defect is selected.

For example, the quantity of the chucks carrying the wafers in the machine is two. In the same batch, 25 wafers are indicated by ID numbers 01, 02, . . . , 25, respectively. The 25 wafers enter the two chucks respectively. For example, the wafers, indicated by ID numbers 01, 03, . . . , 23, 25, enter the chuck indicated by chuck ID 001, and the wafers, indicated by ID numbers 02, 04, . . . , 22, 24, enter the chuck indicated by chuck ID 002. At this time, the wafers indicated by ID numbers 01, 03, . . . , 23, 25 enter the chuck indicated by the same ID. When the number of consecutive occurrences of the hot spot defect information that has the same horizontal and vertical coordinates in the wafers indicated by ID numbers 01, 03, . . . , 23, 25 exceeds a set quantity, in some examples, when there always exist at least three or more wafers that have hot spot defect information having the same horizontal and vertical coordinates in the wafers indicated by ID numbers 01, 03, . . . , 23, 25, at this time it may be determined that the hot spot defect feature of the hot spot defect information is the wafer chuck defect, and then the target hot spot defect information associated with the wafer chuck defect is selected. As shown in FIG. 3, in the wafers indicated by ID numbers 01, 03, . . . , 23, 25, defects at coordinates (−105.333, −70.667) occur on the wafers indicated by ID number 05, 07, and 09. At this time, it is determined that the hot spot defect feature of the hot spot defect information is the wafer chuck defect, and then the target hot spot defect information associated with the wafer chuck defect is selected.

It is to be noted that it may be set that when the hot spot defect information that has the same horizontal and vertical coordinates occurs consecutively at least three times, it is determined that the hot spot defect feature of the hot spot defect information is the wafer chuck defect, or it may be set that the hot spot defect information occurs simultaneously at least four times. The embodiments of the disclosure do not specifically limit the number of times. Those skilled in the art may make specific settings according to the specific accuracy requirements, and the rapid positioning of the target hot spot defect may be realized by setting the number of consecutive occurrences.

In an embodiment, the batch information of each wafer includes process film information and product information.

Since there exist a plurality of process films on the wafer surface, and the wafer needs to enter corresponding process device and be placed on the wafer chuck corresponding to different process device in each process film manufacture, so the batch information of each wafer in the semiconductor manufacturing process further includes process film information. After the target hot spot defect information is acquired, according to the process film corresponding to the target hot spot defect information tracked, the process film where the defect source is located may be found in the process film locked by the target hot spot defect information, thereby tracking and locating the defect source.

Further, the batch information of each wafer further includes the product information. For example, in a process of producing two different products, different products require different processes or process conditions. For example, the wafer used in a first product needs to go through 4 manufacturing processes while the wafer used in a second product needs to go through 3 manufacturing processes.

Further, based on the above-mentioned embodiments, the wafer defect analysis method further includes: the defect source is classified according to the target hot spot defect information, and a corresponding defect source distribution diagram of is drafted according to the process film information of each wafer.

According to the acquired batch information of each wafer in the wafer manufacturing process, the first wafer corresponding to the target hot spot defect information associated with the hot spot defect feature is tracked, and the defect source is tracked. Then the defect source is classified, and a corresponding defect source distribution diagram for the classified defect source is drafted according to the process film information.

Since the quantities of respective process film information are different, and the quantity information of different process film defects may visually reflect the distribution of contaminants at the wafer chuck surface, therefore, after the target hot spot defect information is acquired, the defect quantity information of the target hot spot defect in each process film may be determined.

It is to be noted that the defect quantity information includes quantity information of contamination sources of the target hot spot defect and quantity information of wafers affected by the contamination resource.

As shown in FIG. 6, after the defect quantity information is determined, a one-to-one correspondence between the defect quantity information and the process film information is established so as to achieve visualization analysis of the defect quantity information and the process film information. For example, the respective process film information in a circuit structure manufacturing process at the wafer surface is indicated by A, B, C, D, and E, “particle source by layer” represents quantity information of the contamination sources, and “impact count by layer” represents quantity information of the wafers that are affected by the contamination sources. Different film layer location information corresponds to the quantity information of the defects in the respective film layers. For example, the quantity information of the contamination sources in the film layer A is 20, and the quantity information of the contamination sources in the film layer B is 10, and the quantity information of the subsequent wafers after the corresponding film layer A is affected by the contamination sources is 50 and the quantity information of the subsequent wafers after the film layer B is affected by the contamination sources is 15. The locations of the contamination sources on a certain film layer are tracked by analyzing the quantity information of the contamination sources in the film layer A and the quantity information of the wafers affected by the contamination sources, and a rapid data analysis support is provided for subsequently analyzing the cause of generating defects in the film layers.

Further, the target hot spot defect information further includes the height of the wafer surface hot spot relative to the wafer surface.

In the same film layer manufacturing process, there may be one or more contamination sources on the chuck, and the sizes of the contamination sources are also different, and in the process, due to the adhesion of the back surface of the wafer to the contamination source, the contamination source may be gradually reduced. The characteristic of the target hot spot defect is analyzed through the height of the wafer surface hot spot relative to the wafer surface, which may better reflect the impact of the chuck surface contamination source defect to the wafer. Therefore, visualization analysis is performed to the determined target hot spot defect information by the height of the wafer surface hot spot relative to the wafer surface and the process film information.

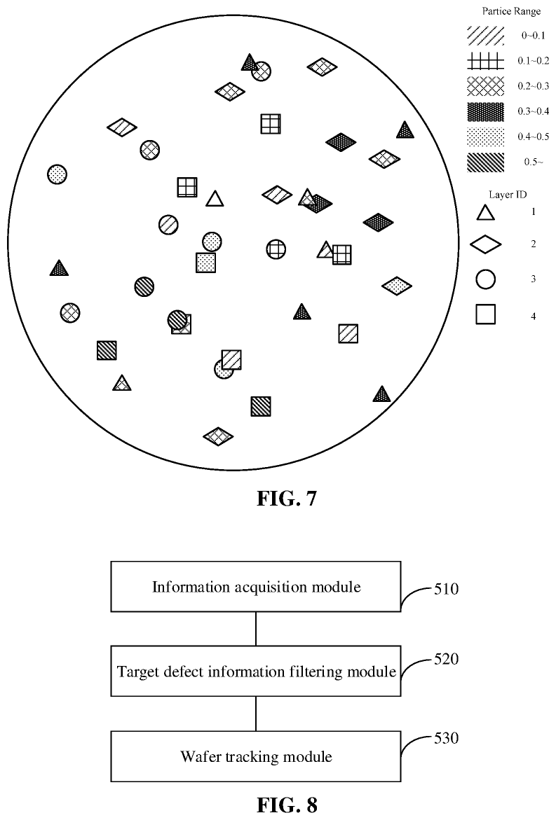

Further, the target hot spot defect information further includes a particle size of the wafer surface hot spot relative to the wafer surface. FIG. 7 is a schematic diagram showing a locational relationship between the particle size of the wafer surface hot spot relative to the wafer surface and the corresponding process film. As shown in FIG. 7, after the defect height and the process film information are determined, a one-to-one correspondence between the defect particle size and the process film information may be achieved. “Layer ID” represents the number of the process film of the wafer, and particle range represents the particle size of the wafer surface hot spot relative to the wafer surface in the process film. According to the acquired process film information, the process film number and the particle size of the wafer surface hot spot in the respective film layer relative to the wafer surface are represented in a visual manner so as to reflect the corresponding relationship between the film layer information and the particle range. In some examples, when the target hot spot defect information in visual graphics appears as a triangle, it may be reflected the particle size of the respective hot spot at the wafer surface relative to the wafer surface in a first process film, and visual tracking for the contamination sources may be realized by analyzing the process film defect information and defect particle size information.

On the other hand, it is also possible to represent the particle size of the wafer surface hot spot relative to the wafer surface in the process film by different colors, a white color represents the defect particle size of 0 μm to 0.1 μm, a red color represents the defect particle size of 0.2 μm to 0.3 μm, and a black color represents the defect particle size of not less than 0.5 μm. The process film location is represented in shape, and a triangle represents a first process film, a circle represents a second process film, and a square represents a third process film. The embodiments of the disclosure do not specifically limit the expression of the corresponding relationship between the defect size and the location information of the process film.

It is to be noted that defects having the same particle size may exist in different film layers or in the same film layer.

Based on the above-mentioned embodiments, FIG. 8 is a schematic structural diagram of a wafer defect analysis system provided by still another embodiment of the disclosure. As shown in FIG. 8, the wafer defect analysis system includes: an information acquisition module 510, configured to acquire batch information and defect information of each wafer in a manufacturing process, the defect information including hot spot defect information; a target defect information filtering module 520, configured to filter out target hot spot defect information associated with the hot spot defect feature from the hot spot defect information according to a set hot spot defect feature; and a wafer tracking module 530, configured to track, according to the batch information, a first wafer corresponding to the target hot spot defect information associated with the hot spot defect feature, and determine a defect source.

Through the wafer defect analysis system provided by the embodiments of the disclosure, the information acquisition module acquires the batch information and the defect information of each wafer in the manufacturing process, the defect information being the hot spot defect information; the target defect information filtering module selects the target hot spot defect information associated with the hot spot defect feature from the hot spot defect information according to the set hot spot defect feature; the wafer tracking module tracks the first wafer corresponding to the target hot spot defect information associated with the hot spot defect feature according to the batch information and determines the defect source, such that the target hot spot defect information associated with the hot spot defect feature is selected from the hot spot defect information by the set hot spot defect feature and the first wafer corresponding to the target hot spot defect information is finally determined, so as to improve the accuracy for tracking the wafer defect source.

Further, as shown in FIG. 9, the wafer defect analysis system further includes a defect source classification module 540, which is configured to classify the defect source according to the target hot spot defect information, and make draft a corresponding defect source distribution diagram according to the batch information of each wafer.

The wafer defect analysis system provided by the embodiments of the disclosure may execute the wafer defect analysis method provided by any of the embodiments of the disclosure and has corresponding functional modules for executing the method and beneficial effects.

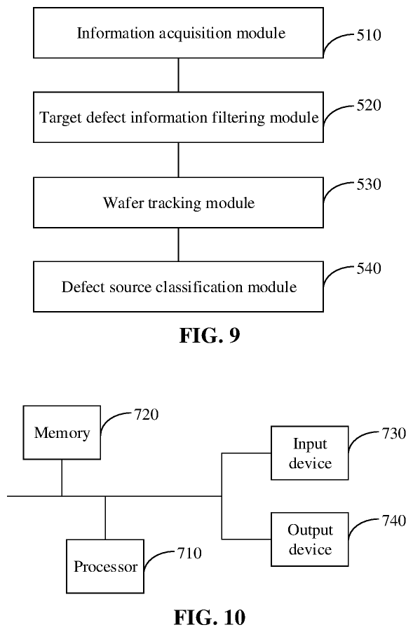

Based on the above-mentioned embodiments, FIG. 10 is a schematic structural diagram of an electronic device provided by still another embodiment of the disclosure. As shown in FIG. 10, the electronic device includes a processor 710, a memory 720, an input device 730, and an output device 740; the quantity of the processor 710 in the electronic device may be one or more. In FIG. 10, one processor 710 is taken as an example. The processor 710, the memory 720, the input device 730, and the output device 740 in the electronic device may be connected by a bus or other ways. In FIG. 10, the bus connection is taken as an example.

The memory 720, as a computer readable storage medium, may be configured to store a software program, a computer executable program, and a module, such as program instructions/modules corresponding to the wafer defect analysis method in the embodiments of the disclosure. The processor 710 runs a software program, an instruction, and a module stored in the memory 720, and thus executes various functional applications of the electronic device and data processing, that is, the wafer defect analysis method provided by the embodiments of the disclosure is realized.

The memory 720 may mainly include a program storage region and a data storage region. The program storage region may store an operating system, an application required for at least one function. The data storage region may store data created according to the use of a terminal. Further, the memory 720 may include a high speed random access memory and may also include a non-volatile memory, such as at least one disk storage device, a flash device, or another non-volatile solid state storage device. In some embodiments, the memory 720 may further include a memory that is remotely set relative to the processor 710. These remote memories may be connected to the electronic device over a network. Embodiments of the above-mentioned network include, but are not limited to, Internet, corporate internal networks, local area networks, mobile communication networks, and combinations thereof.

The input device 730 may be configured to receive input digital or character information, and generate key signal inputs related to user settings and functional control of the electronic device and may include a keyboard, a mouse, or the like. The output device 740 may include a display device such as a display.

Based on the above-mentioned embodiments, the embodiment also provides a storage medium including a computer executable instruction. The computer executable instruction is configured to implement wafer defect analysis method provided in the embodiments of the disclosure when executed by the computer processor.

Of course, a storage medium including a computer executable instruction is provided in the embodiments of the disclosure. The computer executable instruction of the storage medium is not limited to the method operation as mentioned above, and the correlation operation in the wafer defect analysis method provided by any embodiment of the disclosure may also be executed.

Those skilled in the art will become apparent from the description related to the above embodiments that, the disclosure may be implemented by means of software and necessary universal hardware. Of course, the disclosure may be implemented by hardware, but in many cases, the former is a better implementation. Based on this understanding, the embodiments of the disclosure essentially may be embodied in the form of software products. The computer software product may be stored in the computer readable storage medium, such as a floppy disk, a Read-Only Memory (ROM), a Random Access Memory (RAM), a flash, a hard drive or disc, etc., of the computer, including several instructions to make a computer device (may be a personal computer, a server, or a network device, etc.) execute the method described in the respective embodiments of this disclosure.

It is to be noted that in the embodiment of the above-mentioned search device, the respective units and modules included are only divided according to functional logic, and are not limited to the above division, as long as the corresponding function can be achieved.

What is claimed is:

1. A wafer defect analysis method, comprising:
acquiring batch information and defect information of each wafer in a semiconductor manufacturing process, the defect information comprising hot spot defect information;
setting a hot spot defect feature, and selecting target hot spot defect information associated with the hot spot defect feature from the hot spot defect information; and
tracking, according to the batch information, a first wafer corresponding to the target hot spot defect information associated with the hot spot defect feature, and determining a defect source.

2. The wafer defect analysis method of claim 1, wherein the hot spot defect information comprises horizontal and vertical coordinates of a wafer surface hot spot.

3. The wafer defect analysis method of claim 2, wherein the batch information of the each wafer comprises wafer chuck information corresponding to the each wafer, wherein selecting the target hot spot defect information associated with the hot spot defect feature from the hot spot defect information comprises:
selecting, according to the wafer chuck information, the hot spot defect information corresponding to a same wafer chuck;
selecting, in the hot spot defect information corresponding to the same wafer chuck, hot spot defect information having same horizontal and vertical coordinates; and
in a case where a number of consecutive occurrences of the hot spot defect information having the same horizontal and vertical coordinates exceeds a set quantity, determining that the hot spot defect feature of the hot spot defect information is a wafer chuck defect, and selecting target hot spot defect information associated with the wafer chuck defect.

4. The wafer defect analysis method of claim 3, wherein the set quantity is 3.

5. The wafer defect analysis method of claim 1, wherein the batch information of the each wafer comprises process film information or product information.

6. The wafer defect analysis method of claim 5, wherein the wafer defect analysis method further comprises:
classifying the defect source according to the target hot spot defect information, and drafting a corresponding defect source distribution diagram according to the process film information or the product information of the each wafer.

7. The wafer defect analysis method of claim 5, wherein after selecting the target hot spot defect information associated with the hot spot defect feature, the method further comprises:
determining defect quantity information of the target hot spot defect in each process film.

8. The wafer defect analysis method of claim 7, wherein the defect quantity information comprises quantity information of contamination sources of the target hot spot defect and quantity information of wafers affected by the contamination sources.

9. The wafer defect analysis method of claim 7, wherein after determining the defect quantity information, the method further comprises:
establishing a one-to-one correspondence between the defect quantity information and the process film information to achieve visualization analysis of the defect quantity information and the process film information.

10. The wafer defect analysis method of claim 2, wherein the target hot spot defect information further comprises a height of the wafer surface hot spot relative to a wafer surface.

11. The wafer defect analysis method of claim 2, wherein the target hot spot defect information further comprises a particle size of the wafer surface hot spot relative to a wafer surface.

12. The wafer defect analysis method of claim 1, wherein acquiring the batch information and the defect information of the each wafer in the semiconductor manufacturing process comprises: collecting manufacturing information of the each wafer, wherein the manufacturing information at least comprises the batch information containing a number of the each wafer, a batch number of the each wafer, a machine number and a wafer chuck number, together with the hot spot defect information obtained from detection after corresponding process steps are executed on wafer surface of the each wafer.

13. An electronic device, comprising:
at least one processor; and
a storage device, configured to store at least one program;
wherein when executing the program, the processor is configured to:
acquire batch information and defect information of each wafer in a semiconductor manufacturing process, the defect information comprising hot spot defect information;
set a hot spot defect feature, and selecting target hot spot defect information associated with the hot spot defect feature from the hot spot defect information; and
track, according to the batch information, a first wafer corresponding to the target hot spot defect information associated with the hot spot defect feature, and determine a defect source.

14. The electronic device of claim 13, wherein the hot spot defect information comprises horizontal and vertical coordinates of a wafer surface hot spot.

15. The electronic device of claim 14, wherein the batch information of the each wafer comprises wafer chuck information corresponding to the each wafer, wherein when selecting the target hot spot defect information associated with the hot spot defect feature from the hot spot defect information, the processor is configured to:
select, according to the wafer chuck information, the hot spot defect information corresponding to a same wafer chuck;
select, in the hot spot defect information corresponding to the same wafer chuck, hot spot defect information having same horizontal and vertical coordinates; and
in a case where a number of consecutive occurrences of the hot spot defect information having the same horizontal and vertical coordinates exceeds a set quantity, determine that the hot spot defect feature of the hot spot defect information is a wafer chuck defect, and select target hot spot defect information associated with the wafer chuck defect.

16. The electronic device of claim 15, wherein the set quantity is 3.

17. The electronic device of claim 13, wherein the batch information of the each wafer comprises process film information or product information.

18. The electronic device of claim 17, wherein the processor is further configured to:
classify the defect source according to the target hot spot defect information, and draft a corresponding defect source distribution diagram according to the process film information or the product information of the each wafer.

19. The electronic device of claim 17, wherein the processor is further configured to:
defect quantity information of the target hot spot defect in each process film.

20. A non-volatile computer readable storage medium, having a computer program stored thereon, wherein when executed by a processor, the computer program causes the processor to:
acquire batch information and defect information of each wafer in a semiconductor manufacturing process, the defect information comprising hot spot defect information;
set a hot spot defect feature, and selecting target hot spot defect information associated with the hot spot defect feature from the hot spot defect information; and
track, according to the batch information, a first wafer corresponding to the target hot spot defect information associated with the hot spot defect feature, and determine a defect source.
